(19) United States
(12) Patent Application Publication
(10) Pub. No.: US 2007/0065839 A1
(43) Pub. Date: Mar. 22, 2007

(54) Method of identifying agents to prevent or treat diabetes

(76) Inventors: Alan D. Attie, Madison, WI (US); Mark P. Gray-Keller, Middleton, WI (US); Hong Lan, Edison, NJ (US); Philipp W. Raess, Madison, WI (US)

Correspondence Address: QUARLES & BRADY LLP, FIRSTAR PLAZA, ONE SOUTH PINCKNEY STREET, P.O. BOX 2113 SUITE 600, MADISON, WI, 53701-2113, US

(21) Appl. No.: 11/385,571

(22) Filed: Mar. 21, 2006

Related U.S. Application Data
(60) Provisional application No. 60/663,949, filed on Mar. 21, 2005.

(51) Int. Cl. C12Q 1/68; C12Q 1/66

(52) U.S. Cl. 435/6; 435/8

(57) ABSTRACT

The invention provides an assay for identifying agents that can prevent and treat diabetes. Specifically, the assay is a novel tool for identifying agents that can up regulate cholecystokinin (CCK) expression in pancreatic islets of mammals. The increased expression of CCK triggers an increase in pancreatic β-cell mass and plasma insulin levels, which have been found to protect against the onset of diabetes. Also, disclosed are representative therapeutic agents identified through the assay of the invention. The methods of the invention are efficient and readily amenable to high-throughput drug screening protocols.

### CROSS-REFERENCE TO RELATED APPLICATIONS

This application claims the benefit of U.S. Provisional Application No. 60/663,949 filed Mar. 21, 2005. This application is incorporated herein by reference in its entirety.

### STATEMENT REGARDING FEDERALLY SPONSORED RESEARCH OR DEVELOPMENT

This invention was made with United States government support awarded by NIH NIDDK 58037 and NIH NIDDK 66369. The United States government has certain rights in this invention.

### BACKGROUND OF THE INVENTION

About 15 million Americans suffer from type II diabetes mellitus. This disease involves an impaired response to insulin (insulin resistance) and the failure of pancreatic β-cells to compensate with sufficient insulin to titrate blood glucose. Obesity is a strong risk factor for the development of type II diabetes. However, only about 20% of obese people develop diabetes; most obese people can maintain euglycemia (normal blood sugar) throughout their life span despite becoming insulin resistant. Genetic factors play a role in determining whether an obese individual goes on to develop type II diabetes. Therefore, it is believed that diet and obesity collaborate with genetics to produce diabetes.

To understand the differences underlying the two types of obesity; that which resists the onset of diabetes and that which is linked to diabetes, our laboratory previously used DNA micro arrays and RT-PCR to compare gene expression profiles of non-diabetic and diabetic obese mice models. Using this strategy, our lab was able to pinpoint risk factors for developing diabetes by identifying key genes whose expression was altered. Our lab was able to show non-diabetic obese mice maintained euglycemia along with increasing hepatic steatosis, whereas diabetic obese mice had no fatty liver but were severely diabetes. From these results, our lab hypothesized that resistance to diabetes correlates with a high level of hepatic lipogenic gene expression and hepatic steatosis in non-diabetic obese mice. Accordingly, it is believed that increased hepatic lipogenic capacity protects non-diabetic obese mice against the development of type II diabetes. (See, Lan et al., Diabetes, 52:688-700 (2003), which is incorporated by reference herein in its entirety.)

Furthermore, based on the outcome of this gene expression analysis for non-diabetic and diabetic obese mice, our lab observed that the expression of cholecystokinin (CCK), a satiety hormone, one of the many genes that was found to be differentially expressed, dramatically increased in obese mice, regardless of the tendency for the mice to develop diabetes. Accordingly, our laboratory believes that it would be desirable to further examine the role of CCK expression on obesity and diabetes to identify compounds for use in preventing the onset of and treating diabetes in obese individuals.

### BRIEF SUMMARY OF THE INVENTION

The present invention is broadly summarized as an assay method for identifying agents specific for up regulating CCK expression. The assay is based on applicants' recognition that up regulating CCK signaling in pancreatic islets or CCK-producing cells in or near the islets promotes an increase in pancreatic β-cell mass, plasma insulin levels, and glucose-stimulated insulin secretion. This increase is required to maintain glucose homeostatis and thereby protects against the onset of diabetes.

Accordingly, in one aspect, the invention provides a method for identifying an agent effective for up regulating CCK by performing a screening assay. The assay includes the steps of providing an experimental reporter expression vector having a CCK promoter operably linked to an experimental reporter gene; providing a control reporter expression vector having a control promoter operably linked to a control reporter gene; wherein the control reporter gene and the experimental reporter gene are separately detectable; co-transforming the experimental vector and the control vector in host cells; exposing the co-transformed cells to a candidate CCK up-regulating agent, such that cells affected by the agent exhibit an increased signal intensity; measuring the signal intensity exhibited by each reporter gene sequentially from a single cell culture sample; and identifying an effective CCK up-regulating agent based on an increase in the experimental to control reporter expression signal intensity ratio.

In a related aspect the host cells are pancreatic islet cells or cells derived from pancreatic islets, such as an immortalized β-cell line.

Yet another aspect, the assay is a high throughput screening assay.

In a related aspect, the invention provides a nucleic acid construct having a CCK promoter operably linked to an experimental reporter gene, preferably firefly luciferase, wherein the construct is an experimental reporter expression vector.

In a related aspect, the invention provides a nucleic acid construct having a control promoter operably linked to a control reporter gene, preferably Renilla luciferase, wherein the construct is a control reporter expression vector.

In another aspect, the invention provides a kit containing a nucleic acid construct having a CCK promoter operably linked to an experimental reporter gene, preferably firefly luciferase.

In another aspect, the invention provides a kit containing a nucleic acid construct having a control gene promoter operably linked to a control reporter gene, preferably Renilla luciferase.

In a related aspect, the invention provides a kit for identifying an agent effective for up regulating CCK. The kit having (i) a nucleic acid construct having a control gene promoter, optionally beta-actin or other constitutively expressed house-keeping gene, operably linked to a control reporter gene, wherein the construct is a control reporter expression vector; and (ii) nucleic acid construct having a CCK promoter operably linked to a experimental reporter gene, wherein the construct is a experimental reporter expression vector; and b) instructions for use.

In another aspect, the invention provides methods for preventing or treating diabetes in a mammal in need thereof, by administering to the mammal a CCK up regulating agent identified by using the assay method described herein, wherein the agent is capable of preventing or treating diabetes, and wherein the agent is administered in an effective amount sufficient to increase the expression of CCK in pancreatic islet cells relative to islet cells of mammals not having been exposed to the agent.

One feature of this aspect is that the increased CCK expression in pancreatic islet cells promotes an increase in β-cell mass, plasma insulin levels, or potentiates glucose-stimulated insulin secretion.

Another feature is that the increased CCK expression in pancreatic islet cells is localized and not systemic.

In another aspect, the invention provides representative agents, CCK promoter agonists identified by using the assay method described herein. The agents capable of up regulating CCK signaling in pancreatic islets or CCK producing cells in or near the islets, to promote an increase in pancreatic β-cell mass, plasma insulin levels, or promoting glucose-stimulated insulin secretion.

In this aspect, the upregulation in CCK expression in pancreatic islet cells is localized and not systematic.

Also, in this aspect, the agents, upregulators of CCK expression, are effective at increasing β-cell mass or preventing or delaying the onset of diabetes.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used.

Other objects, advantages and features of the present invention will become apparent from the following specification taken in conjunction with the accompanying drawings.

### BRIEF DESCRIPTION OF THE SEVERAL VIEWS OF THE DRAWINGS

FIG. 1 is a graph showing upregulation of CCK in pancreatic islets.

FIG. 2 is a scatterplot showing that deletion of CCK causes mice to display relative hypoinsulinemia.

FIGS. 3A-B are photographs showing that CCK^{null} B6-ob/ob mice have smaller islets.

FIG. 4 is a scatterplot showing that B6-ob/ob-CCK^{null} mice have higher frequency of β-cell apoptosis than B6-ob/ob-CCK^{wT} mice.

FIG. 5 is graph showing human islet CCK expression and body mass index.

FIGS. 6A-B show nucleic acid constructs used in the screening assay described herein. (A) an experimental reporter expression vector having a 20 kb CCK mouse promoter operably linked to an experimental reporter gene; (B) an experimental reporter expression vector having a 12 kb CCK mouse promoter operably linked to an experimental reporter gene.

### DETAILED DESCRIPTION OF THE INVENTION

The present invention broadly relates to methods for identifying therapeutic agents used in the prevention and treatment of diabetes. Specifically, the present invention provides a novel screening assay for identifying agents specific for up regulating CCK expression in mammals. The assay is based on applicants' recognition that up regulating CCK signaling in pancreatic islets or CCK producing cells in or near the islets promotes an increase in pancreatic β-cell mass, plasma insulin levels, or glucose-stimulated insulin secretion relative to non-upregulated cells. This increase is required to maintain glucose homeostatis and thereby protects against the onset of diabetes.

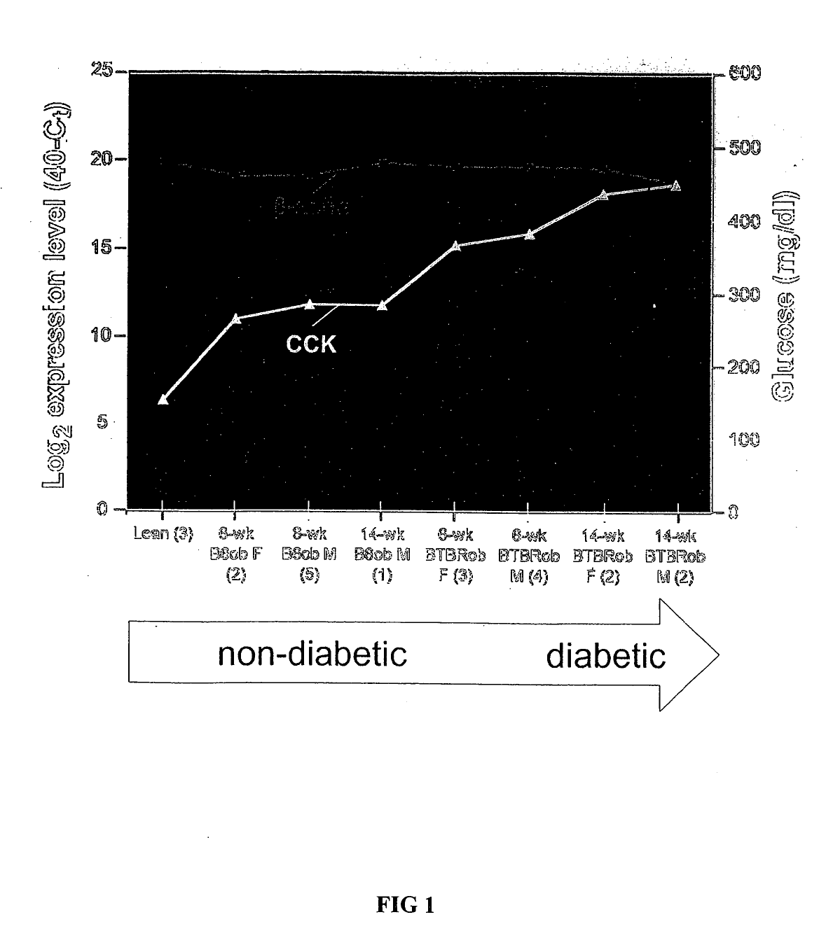

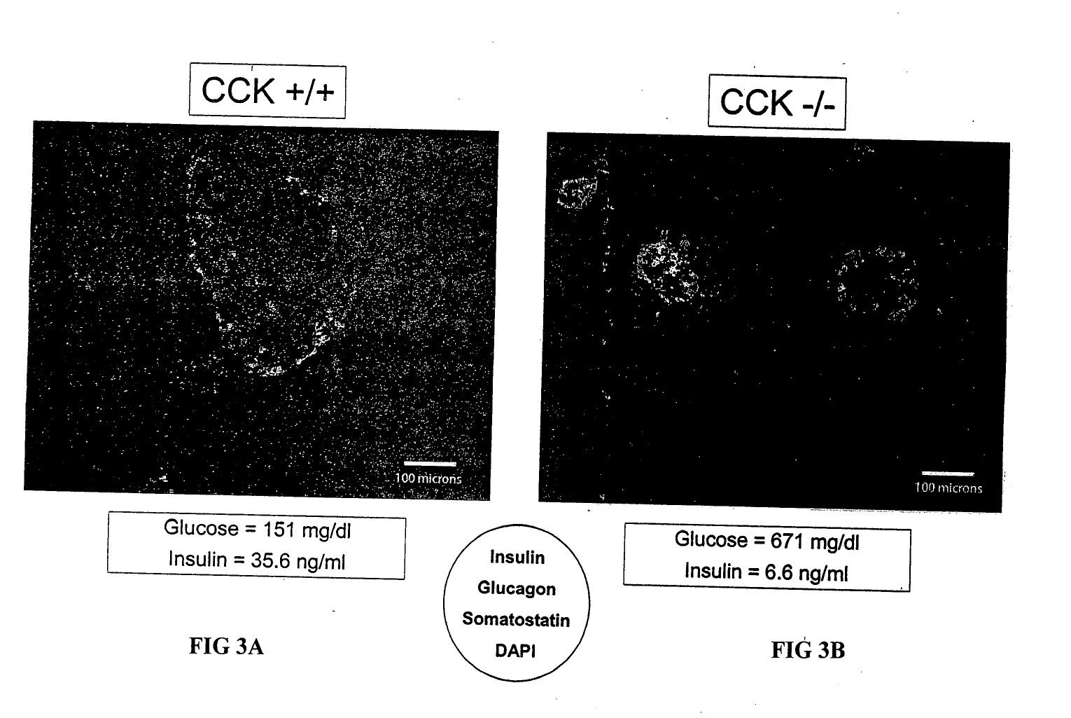

The recognition that up regulating CCK signaling in pancreatic islets causes a biochemical cascade that protects against the onset of diabetes came about through a series of recent experiments conducted by the applicants. The expression of CCK was dramatically elevated in pancreatic islets of genetically obese (ob/ob) C57BL/6 (B6) mice, as shown for example in FIG. 1. It was also observed that despite severe insulin-resistance, these mice did not develop diabetes, due to a significant increase in plasma insulin levels and β-cell mass, as shown, for example, in FIG. 3. Based on these experimental observations, applicants hypothesized that the increase in CCK expression acts as an anti-diabetogenic signal, and is essential for preventing obese mice from developing diabetes. Accordingly, applicants predicted that an increase in CCK must be associated with preventing the onset of diabetes in obese mice. Similarly, applicants predicted that in the absence of CCK, the obese B6-ob/ob mice would be more susceptible to developing diabetes because there would be a corresponding decrease in plasma insulin levels and β-cell mass.

Alternatively, by conducting additional preliminary experiments using CCK deficient mice, applicants were able to determine that the obese mice without the CCK gene would have decreased beta-cell mass and plasma insulin levels. To conduct these experiments, applicants designed B6-ob/ob-CCK^{null} mice, by introgression of a CCK^{null} allele into the B6-ob/ob mice. Specifically, to produce B6-ob/ob-CCK^{null} mice, B6 mice heterozygous for the leptin gene (ob/+, homozygous ob/ob mice are sterile and cannot breed) were crossed with B6 mice homozygous for the CCK null gene. All of the offspring that were heterozygous for the CCK null allele were typed for the ob gene by SSCP (single stranded confirmational polymorphism), which can detect a one base pair mutation. The mice that were ob/+were crossed with each other.

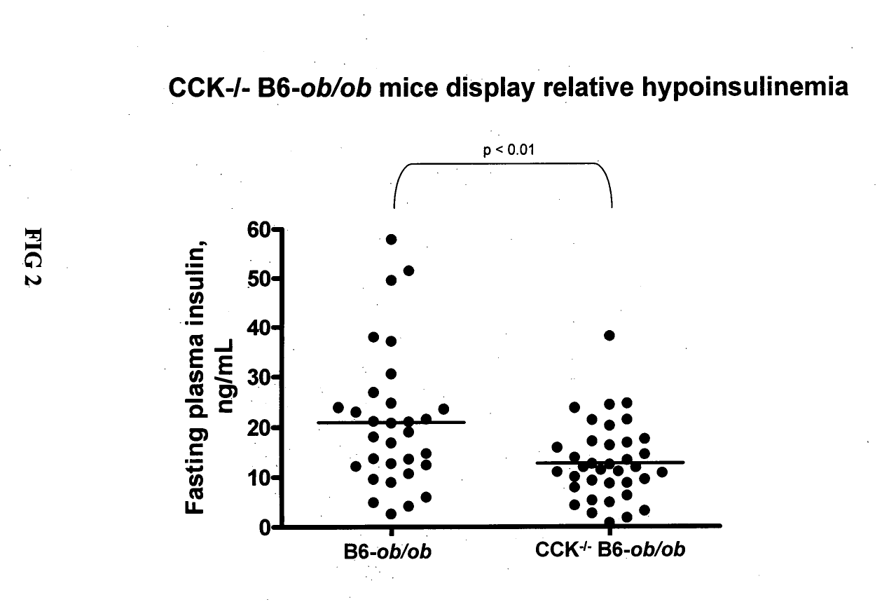

The resulting offspring were then typed for ob (by SSCP) and for CCK by amplifying a DNA fragment that spanned the normal CCK gene and the fragment that had been inserted to disrupt the gene. Only those that had the null gene showed a band of that size. Normal CCK genes were detected by amplifying a fragment of normal DNA, which would only amplify if the gene were uninterrupted. One in eight mice were homozygous null and ob/+; these were then crossed to produce mice all of which would be CCK null, one quarter of which would also be ob/ob. Applicants observed that in contrast to the mice that were genetically obese and had elevated CCK expression, ob/ob mice that are also deficient for CCK had decreased beta-cell mass and plasma insulin. Specifically, FIG. 2 shows that deletion of CCK causes mice to display relative hypoinsulinemia.

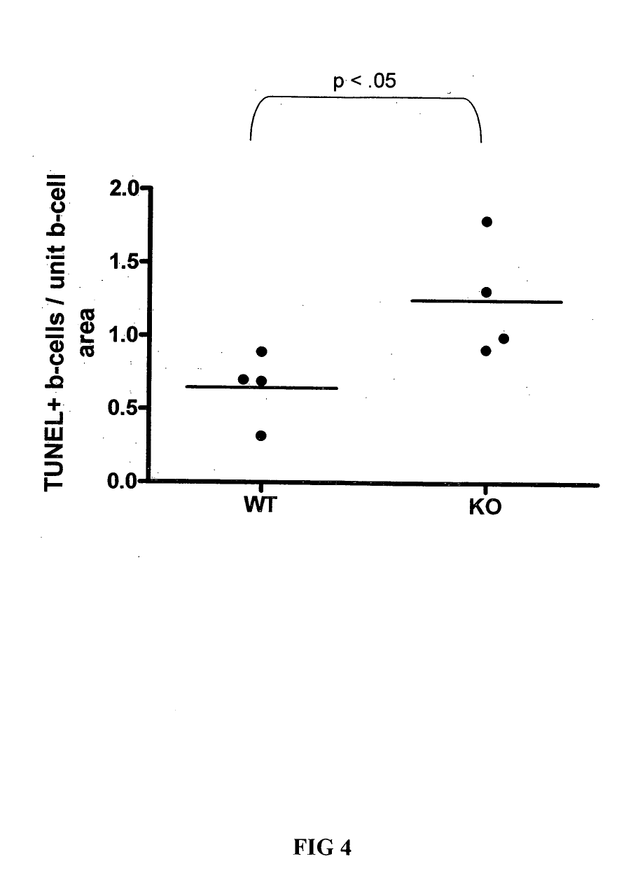

Further investigation into the phenotype of B6-ob/ob-CCK^{null} mice revealed that loss of CCK expression promotes an increase in beta-cell apoptosis as shown in FIG. 4. This suggests that upregulation of CCK expression prevents beta-cell apoptosis. Accordingly, we believe that this may lead to the mechanism underlying the observed loss of beta-cell mass in B6-ob/ob-CCK^{null} mice.

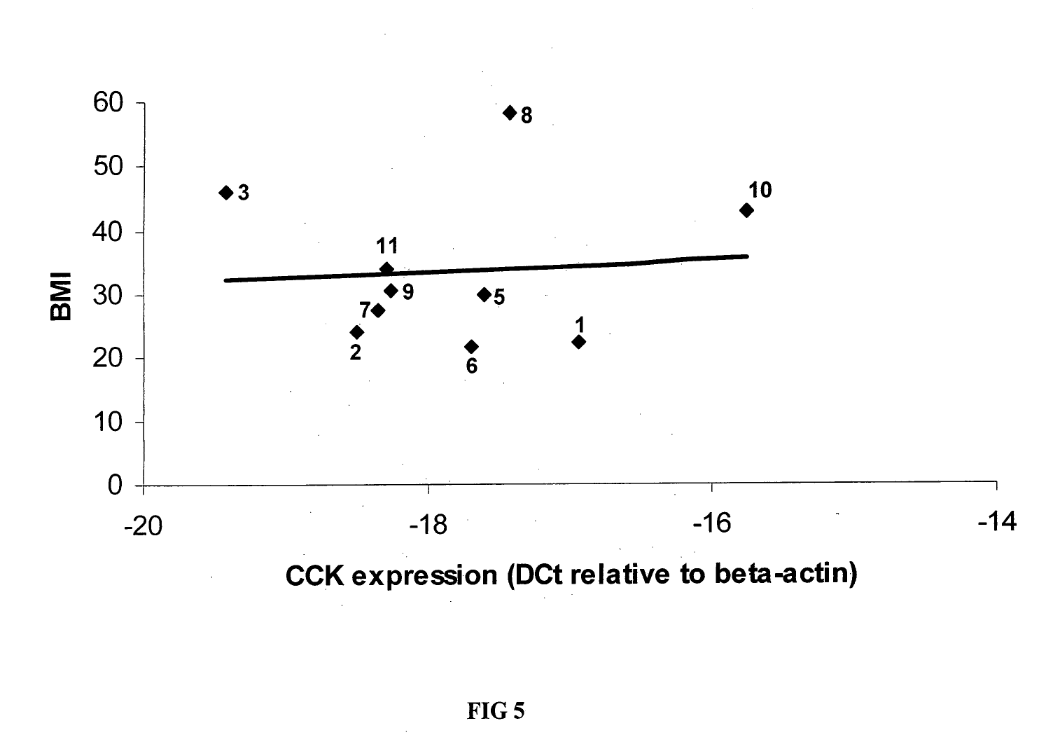

Additional studies were conducted to determine the levels of islet CCK expression in lean as compared to obese humans. FIG. 5 reveals that there is no correlation between obesity and islet CCK expression, which we see in mice. Furthermore, absolute islet CCK expression levels are low in all human subjects. This suggests that upregulation of CCK expression in human islets is possible and that the effects of islet CCK upregulation on beta-cell mass and plasma insulin levels are not already saturated in obese humans. It is also contemplated that CCK is acting in a paracrine manner; i.e., cells that do not up regulate CCK may be affected by increased CCK in neighboring cells. Thus, up regulating islet CCK expression in humans may have beneficial effects on beta-cell mass and plasma insulin levels in obese subjects. These results taken together demonstrate that CCK signaling promotes an increase in pancreatic β-cell mass, which is required for maintaining glucose homeostasis and preventing the onset of diabetes in obese individuals.

Accordingly, based on applicants' results, it is believed that agents effective for up regulating CCK signaling in pancreatic islets provide a novel therapeutic approach for the prevention and treatment of diabetes. It is also envisioned that such agents could stimulate CCK signaling to promote β-cell proliferation or block β-cell apoptosis in vitro. Such increase in the number of β-cells results in an increase in the number of β-cells available for use in treatment of diabetes, such as for example, in pancreatic islet transplantation.

Applicants believe that by screening compound libraries, suitably effective agents (i.e., agonists) can be identified which are capable of increasing CCK expression and/or secretion when applied to pancreatic islet cells. In addition to pancreatic islets, it is encompassed that other cell lines derived from islets including, not limited to those derived from β, α, δ, PP or ghrelin-containing ε-cells; or cells as yet unidentified CCK-producing cells in or near pancreatic islets would be equally applicable for use in identifying potentiators of CCK signaling. Based on applicants' preliminary results it is believed that such agonists of CCK signaling in pancreatic islets offer a promising approach for the prevention and treatment of diabetes by promoting an endogenous pathway, which is capable of triggering an increase in β-cell proliferation or decrease in β-cell apoptosis.

Thus, applicants have designed an assay to screen for agents capable of increasing CCK expression and/or secretion when applied to pancreatic islet cells or cells derived therefrom as described herein. In one embodiment, the invention provides a screening assay that may be performed by stably co-transforming suitable cells (such as pancreatic islet cells) with reportable expression vectors. It is contemplated that one reporter construct would serve as an internal control and another reporter construct would serve as an experimental vector containing the CCK promoter operably linked to a reporter gene. In constructing the vectors, the promoter for the gene of interest, such as the CCK promoter is operably linked to an experimental reporter gene through standard recombinant DNA techniques (see, Ausubel et al., Current Protocols in Molecular Biology (John Wiley & Sons, Inc., New York, 1999)).

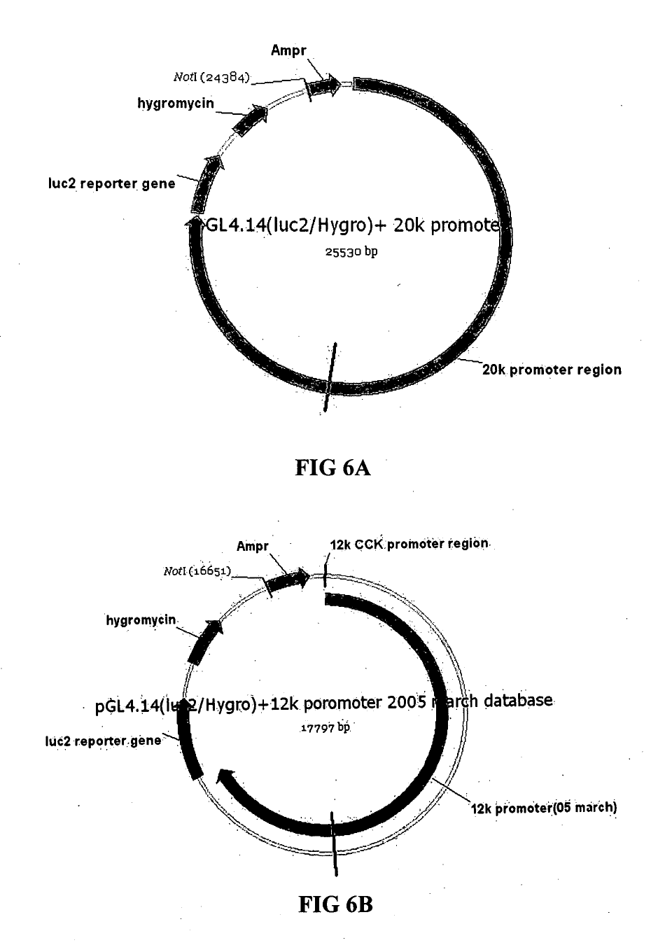

Preferred vectors of the invention are designed so as to integrate the assays of two separate detectable reporter systems, such that one vector would have an experimental reporter and the other would contain a normal control reporter. Accordingly, in one embodiment, the invention provides a nucleic acid construct having a CCK promoter operably linked to an experimental reporter gene, preferably firefly luciferase, wherein the construct is an experimental reporter expression vector. This nucleic acid construct is described in FIG. 6 (A) mouse CCK promoter, 20 kb in length (SEQ ID NO:1); and (B) mouse CCK promoter, 12 kb in length (SEQ ID NO:2) immediately upstream of the mouse CCK gene. It is believed that the 20kb construct contains all the domains necessary to up regulate CCK expression. However, smaller portions of the promoter region immediately upstream of the CCK gene maybe used for practicing the invention and identifying agents for effectively up regulating CCK expression.

It is also contemplated that varying lengths of mammalian CCK promoters (preferably mouse or human) and preferably immediately upstream of the CCK gene maybe operably linked to the reporter gene and used to carry out the assay of the invention.

The assay can be used as a tool to determine which portions of the CCK promoter are needed to regulate CCK expression. This can readily be done by one skilled in the art by preparing promoter deletion constructs, where progressively smaller pieces of the promoter are created, operably linked to a suitable reporter gene and used in the assay.

In a related embodiment, the invention provides a nucleic acid construct having a control promoter operably linked to a control reporter gene, preferably Renilla luciferase, wherein the construct is a control reporter expression vector. Preferably the expression of both reporter proteins could be measured from a single sample.

Once the suitable islet cells are stably co-transformed with reportable expression vectors, the cells are grown to a suitable state of confluency in microtiter wells. The cells in the wells are contacted with various test compounds from a compound library. As used herein the term “test compound” is also referred to as “candidate CCK up-regulating agent.” These agents are randomly screened from either commercially available or private small molecule compound libraries.

After an incubation period that is sufficient to demonstrate a measurable signal in the assay system, the signal level for the experimental and control reporters are measured accordingly to standard techniques known to those of skill in the art. Specifically, the wells containing varying proportions of candidates are then evaluated for signal activation. Based on the comparison of the ratio of experimental to control reporter protein expression, representative candidates that would increase CCK gene expression, are then selected for further evaluation as clinical therapeutic agents for preventing the onset of diabetes in obese individuals. In a preferred embodiment, an increase in the experimental reporter expression as compared to that of the control reporter protein would be considered a positive “hit.”

In a preferred embodiment, the invention provides a method for identifying an agent effective for up regulating CCK by performing a screening assay. The assay includes the steps of providing an experimental reporter expression vector having a CCK promoter operably linked to an experimental reporter gene; providing a control reporter expression vector having a control promoter operably linked to a control reporter gene; wherein the control reporter gene and the experimental reporter gene are separately detectable.

The experimental vector and the control vector are co-transformed in host cells. The co-transformed cells are then exposed to a CCK up-regulating agent, such that cells affected by the agent exhibit an increased signal intensity; measuring the signal intensity exhibited by each reporter gene sequentially from a single cell culture sample. An effective CCK up-regulating agent can then be identified based on an increase in the experimental-to-control reporter expression signal intensity ratio.

As used herein, the term “cholecystokinin” or “CCK” refers to a gastrointestinal hormone that is utilized by the body in the cascade of events which are part of hunger, eating, digestion, satiety and gall bladder contraction. Although CCK has a variety of regulatory roles in the body, it is important for control of pancreatic enzyme secretion. (See, Crawley J.N. et al., Peptides, (1994) 15:4, 731-755, incorporated by reference herein in its entirety). The CCK gene is well characterized from a variety of species, including mammals. Mouse and human genes are highly homologous, especially in the portion of the gene that encodes the bioactive form of CCK. The NCBI accession numbers for the mouse CCK gene are NM_{—}031161 and BC028487. The human CCK gene accession Nos. are NM_{—}000729, BC111026 and BC008283.

More specifically, the entire CCK gene is characterized from both mouse CCK (Vitale et al., (1990) Nucleic Acids Res 19:169-177, incorporated by reference in its entirety), and human CCK (Takahashi et al. (1986) Structure of human cholecystokinin and its chromosomal location. Gene 50:353-360, incorporated by reference in its entirety).

As used herein the term “CCK Promoter” refers to a sequence upstream of the CCK gene that regulates the CCK Gene Expression. The promoter regions of both mouse and human CCK genes are well-characterized. The mouse CCK gene promoter region possess the same four well characterized transcriptional control elements as the human CCK gene, namely an E-box, AP-1 binding site, Sp1 site, and TATA box. (See Rourke et al., (1997) Endocrinology Vol. 138, No. 4 1719-1727). It has also been reported that USF, Sp1, and members of the CREB/ATF and AP-1 family of transcription factors are the major determinants of CCK gene transcription. (see Nielson et al., (1996) DNA Cell Biol. Jan;15(1):53-63). Also, more recently, the cloning of the rat CCK gene has revealed that the promoter contains a number of regulatory elements all located within 100 bp of the TATA box including a putative basic helix-loop-helix leucine zipper element, an SP1 element, and a combined cyclic AMP and TPA response element (see, Hansen et al. (2004) J Neurochem. Apr 89(1):15-23.)

As used herein the term “control gene promoter” refers to any constitutively expressed house-keeping gene, such as for example, beta-actin. Such control gene promoters are widely known in the art.

As used herein, the term “reporter gene” refers to a gene that encode a polypeptide not otherwise produced by the host cell which is detectable by analysis of the cell culture using standard techniques, e.g., by the direct fluorometric, radioisotopic or spectrophotometric analysis of the cell culture. Preferably the gene encodes an enzyme which produces colorimetric or fluorometric changes in the host cell which is detectable by in vitro, in situ or in vivo analysis and which is a quantitative or semi-quantitative function of transcriptional activation. Exemplary enzymes include luciferase, chloramphenicol acetyl transferase, β-galactosidase, secreted placental alkaline phosphatase, human growth hormone, esterases, phosphatases, proteases (tissue plasminogen activator or urokinase) and other secreted enzyme reporters and other enzymes whose function can be detected by appropriate chromogenic or fluorogenic substrates known to those skilled in the art.

In a preferred embodiment, the reporter proteins are luciferases as described hereinbelow. Preferably, the luciferases used in the assay should be distinguishable from one another, if two luciferases are used as the reporter proteins. In a particularly preferred embodiment, one reporter protein is firefly luciferase from Photinus pyralis, and the other is Renilla luciferase from Renilla reniformis. The protein levels may be determined using the Dual-Luciferase Assay System (see Dual-Luciferase Reporter 1000 Assay System, Technical Manual No. 046, Promega Corp., Madison, Wis., 1999; incorporated herein by reference here as if set forth in its entirety).

It is further contemplated and within the scope of this invention that by using the screening assay of the invention, those skilled in the art could easily identify candidate agents or compounds that are suitable for preventing diabetes onset in susceptible individuals, such as those suffering from obesity or related conditions. In this regard, the terms “agent,” “candidate compound,” or “agonist” as used herein, refer to any small molecule that suitably binds with specificity to the CCK peptide hormone promoter, up regulating CCK expression in the pancreatic islets, preferably, so as to increase plasma insulin levels and β-cell mass and prevent the onset of diabetes.

Applicants envision that by using the assay method described herein, those skilled in the art can more readily identify agonists specific for up regulating CCK expression to serve as lead compounds for further pharmaceutical research and development in the field of diabetes.

Accordingly, in another embodiment, the invention provides for representative therapeutic agents capable of up regulating CCK expression in pancreatic islets of mammals. Such agents would serve to trigger a cascade of events leading to an increase in pancreatic β-cell mass, plasma insulin levels, and glucose-stimulated insulin secretion, which would protect against the onset of diabetes. Furthermore, once suitably effective CCK-specific agonists are found, systematic chemical modifications can be made, and their effects can be further assessed using enhanced promoters according to the method of the invention. By following such a systematic development strategy the intrinsic activity of new agonists can be optimized so as to be useful therapeutically against preventing the onset of diabetes.

Similarly, knowing that a particular agent functions as an agonist facilitates identifying which agent is most likely to achieve a given physiological effect, or to achieve a physiological effect absent an unwanted side effect. Thus, in another embodiment, the invention encompasses a method for the treatment or prevention of a diabetes involving CCK that includes administering to a mammal, preferably a human, a therapeutically effective amount of an agent that upregulates CCK expression, identified through the assay described hereinbelow. It is also contemplated that agents identified through the assay described herein could be administered in combination with other compounds that for prevention or treatment of diabetes.

The following examples are provided as further non-limiting illustrations of particular embodiments of the invention.

### PROPHETIC EXAMPLE

Experimental Design

To identify small molecules that promote CCK expression, applicants envision a screening assay that uses a dual luciferase reporter system, based on the Dual-Luciferase® Reporter (DLR) Assay System designed for HTS applications, commercially available through Promega Corp., (Madison, Wis.). The key feature of this system is that two different luciferase reporters are used, one from firefly (Photinus pyralis) and the other from sea pansy (Renilla reniformis). Firefly and Renilla luciferases have distinct enzyme structures and substrate specificities making it possible to selectively discriminate their respective bio-luminescent reactions in the same sample. Each of the two different luciferase reporter enzymes are expressed simultaneously in each cell. During analysis, they are measured sequentially from a single sample. The firefly luciferase reporter is measured first by adding luciferase assay reagent II (LARII, available through Promega Corp.). The Renilla luciferase reaction is initiated by adding the Stop & Glo reagent, available through Promega Corp.) to quench the first reaction and provide substrate for the Renilla enzyme.

Typically, the experimental reporter is correlated with the effect of specific experimental conditions, while the activity of the co-transfected “control” reporter gene provides an internal control, which serves as the baseline response. Normalizing the experimental reporter gene to the activity of an internal control minimizes the variability caused by differences in cell viability and transfection efficiency. A related feature of this assay system is that because the experimental and control luciferase enzymes have distinct evolutionary origins, they can discriminate between their respective bioluminescent substrates and do not cross-activate.

Specifically, in accordance with the invention, it is envisioned that the firefly-induced luminescence will be used to monitor activity of the CCK promoter (i.e., the “experimental” signal) and Renilla-induced luminescence to provide a signal proportional to cell number, general health, transfection efficiency, etc (i.e., the “baseline” or “control” signal). Applicants believe that by normalizing the experimental signal to the baseline signal, the experimental variability inherent to high throughput screens will be minimized and the ability to identify small molecules that can agonize CCK expression will be maximized. Also, compounds effective at promoting CCK expression will be identified as those that can increase the ratio of signal-to-baseline luminescence.

### Materials and Methods

Expression vectors

The Luciferase expression vectors used in this example (i.e., pGL3 and phRL family of expression vectors) are commercially available through Promega Corp. Utilizing the multiple cloning site placed immediately in front of the luciferase gene, the firefly expression vector (pGL3) will be modified to incorporate the promoter of the mouse CCK gene. Therefore, in the modified plasmid (pGL3), expression of the firefly luciferase will be under the control of the CCK promoter. Representative examples of CCK promoters used in preparing the expression constructs include the 20kb upstream of the CCK gene (SEQ ID NO:1) and the 12kb of DNA sequence upstream from the CCK gene (SEQ ID NO:2). It is contemplated that smaller portions of the CCK promoter immediately upstream of the CCK gene are also effective for practicing the novel assay method.

The phRL family of expression vectors containing the Renilla luciferase gene will be used to provide a high-level of Renilla luciferase expression under the control of the CMV, SV40 or HSV-TK promoter. Each plasmid would contain, in addition to the luciferase gene, a mammalian antibiotic selection marker (neomycin, hygromycin or puromycin). Using commercially available transfection reagents, these two plasmids will be co-transfected and stably expressed in a variety of cell lines identified below, under co-selection of the two antibiotic markers present on the plasmids. Suitable co-transfection and stable expression techniques are widely known and practiced in the biotechnology field.

Cell lines

It is envisioned that a variety of cell lines will be used in conducting the primary screen as well as the secondary screens for identifying lead agents or compounds capable of promoting CCK expression. However, since applicants' current research indicates that within the pancreas, CCK is expressed exclusively in pancreatic islet cells, including β-cells and β-cells, the assay will seek to identify compounds that can promote CCK expression, specifically, in these cells. To identify increased CCK expression in pancreatic islet cells, non-islet cells will also be examined, including cells derived from acinar, macrophages and liver. All cells under consideration would be commercially available.

HTS assay protocol

To identify which agents or agonists will yield an increase in CCK expression in cells stably expressing firefly and Renilla luciferase, applicants envision seeding the transformed cells into 96-well microtiter plates (MTP) and growing the cells to ˜90% confluence in a humidified 37° C. tissue culture incubator. Transformation of cells is a method widely practiced by those skilled in the art. A variety of small molecule compounds will be added to individual wells and grown for an additional 24-48 hrs. On the day of the luciferase assay, the growth medium will be removed and PBS solution will be added to gently wash the cell monolayer. Lysis buffer will be added to each well to lyse the cells. The plate containing the cell lysate would be incubated at room temperature with gently shaking for about 15 minutes to an hour.

After the cells have been lysed, the LARII (luciferase assay reagent II, containing the firefly luciferase-specific substrate) and Stop & Glo reagents (containing a quenching compound specific for firefly luciferase and a Renilla luciferase-specific substrate) are prepared. Both LARII and Stop & Glo reagents are commercially available through Promega Corp. Once this is complete, the lysate and substrate would be incubated and the results read on a luminometer. Specifically, the LARII is added to each well and the luminescence would be read within 2 minutes. The Stop & Glo reagent is then added to each well of the plate and the luminescence read within 2 minutes. The luminescence ratio for the firefly luciferase to that observed for the Renilla luciferase will be taken as a measure of compound (i.e., CCK up regulating agent)-dependent activation of the CCK promoter. Accordingly, the signal intensity exhibited by each reporter gene sequentially from a single cell culture sample will be measured. Potential CCK-specific agonists will yield an increase in the firefly:Renilla luciferase ratio.

This assay protocol is designed to identify an agent effective for up regulating CCK in a specific, fast and convenient manner. The assay protocol may be packaged in a kit format. In addition, this assay may be scaled to accommodate a high through-put format. Thus, the methods of the invention are efficient and readily amenable to high-throughput drug screening protocols. Preferably, the subject assays identify compounds not previously known to have the effect that is being screened for.

Furthermore, it is intended that the kit can include “Instructions for use,” for how to carry out the described assay protocol. The amounts of the various reagents in the kits can be varied depending on a number of factors, such as the optimum sensitivity of the assay. The instructions for use are suitable to enable an analyst to carry out the desired assay.

Accordingly, in one embodiment, the invention provides a kit for identifying an agent effective for up regulating CCK containing (i) a nucleic acid construct having a control promoter operably linked to a control reporter gene, wherein the construct is a control reporter expression vector, and (ii) a nucleic acid construct having a CCK promoter operably linked to a experimental reporter gene, wherein the construct is a experimental reporter expression vector. The kit can optionally include instructions for use.

It is contemplated that this assay kit would be the primary screen to identify specific agents that can affect pancreatic islet β-cells. The identification of preliminary CCK agonists affecting β-cells (i.e., “hits”) would be followed by further characterization in secondary screens, in a variety of additional cell types described herein, including in non-islet cells expressing the two luciferases. Accordingly, only those agents that exhibit β-cell specific activity would be selected as suitable regulators of pancreatic islet β-cells. It is preferred that upregulation of CCK be maintained in the local microenvironment of the pancreatic islet cells, rather than system wide, to prevent pancreatitis and possibly other undesirable conditions resulting from the disease.

It is also contemplated that compounds exhibiting CCK promoter activation will be added to unmodified cells to examine whether CCK expression is upregulated as judged by RT-PCR determination of CCK mRNA. In vivo proof-of-concept studies can be designed once CCK-upregulators have been characterized for efficacy with in vitro models. These in vivo studies will determine if up-regulation of CCK expression in pancreatic islets yields an increase β-cell mass, increased plasma insulin levels, or increased glucose-stimulated insulin secretion.

Those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

We claim:

1. A method of performing a biological assay, the method comprising the steps of:
a) providing an experimental reporter expression vector having a cholecystokinin (CCK) promoter operably linked to an experimental reporter gene;
b) providing a control reporter expression vector having a control promoter operably linked to a control reporter gene; wherein the control reporter gene and the experimental reporter gene are separately detectable;
c) co-transforming the experimental vector and the control vector in host cells;
d) exposing the co-transformed cells to a candidate CCK up-regulating agent, such that cells affected by the agent exhibit an increased signal intensity; and
e) measuring the signal intensity exhibited by each reporter gene sequentially from a single cell culture sample.

2. The method of claim 1 further comprising the step of:
f) identifying an effective CCK up-regulating agent based on an increase in the experimental-to-control reporter expression signal intensity ratio.

3. The method of claim 1 wherein the control reporter gene is Renilla luciferase and the experimental reporter gene is firefly luciferase.

4. The method of claim 1 wherein the host cells are pancreatic islet cells or cells derived from pancreatic islets.

5. The method of claim 4 wherein the derived cells are an immortalized β-cell line.

6. The method of claim 1 wherein the assay is a high throughput screening assay.

7. A CCK up-regulating agent identified through the assay of claim 1.

8. An assay method for identifying an agent effective for up regulating cholecystokinin (CCK), the method comprising the steps of:
a) providing an experimental reporter expression vector having a CCK promoter operably linked to an experimental reporter gene;
b) providing a control reporter expression vector having a control promoter operably linked to a control reporter gene; wherein the control reporter gene and the experimental reporter gene are separately detectable;
c) co-transforming the experimental vector and the control vector in host cells;
d) exposing the co-transformed cells to a candidate CCK up-regulating agent, such that cells affected by the agent exhibit an increased signal intensity;
e) measuring the signal intensity exhibited by each reporter gene sequentially from a single cell culture sample; and
f) identifying an effective CCK up-regulating agent based on an increase in the experimental-to-control reporter expression signal intensity ratio.

9. A nucleic acid construct comprising a cholecystokinin promoter operably linked to an experimental reporter gene, preferably firefly luciferase, wherein the construct is an experimental reporter expression vector.

10. A nucleic acid construct comprising a control gene promoter operably linked to a control reporter gene, preferably Renilla luciferase, wherein the construct is a control reporter expression vector.

11. A kit comprising the nucleic acid construct of claim 9.

12. A kit comprising the nucleic acid construct of claim 10.

13. A kit for identifying an agent effective for up regulating cholecystokinin (CCK) comprising:
(i) a nucleic acid construct having a control promoter operably linked to a control reporter gene, wherein the construct is a control reporter expression vector; and
(ii) nucleic acid construct having a CCK promoter operably linked to a experimental reporter gene, wherein the construct is a experimental reporter expression vector.

14. The kit of claim 13 having instructions for use.


SEQUENCE LISTING (2 sequences)

SEQ ID NO: 1 - DNA, 25530 bp, Mus musculus
  misc_feature (6374)..(6473): n is a, c, g, or t
ggcctaactggccggtacctgagctcaggcagttttgctgtcacggggagggtcacagggctagccttccctcaactggtggcttttactcccctgctgtaggtcactgaccagcttctggtgcactatcgtgtttgggcagcagaagaaaaggcctaaaggctgccttccccatcagcagcaaaaccaccaggctggcttttaatatattcaaagtggaaagacacttgaccttctcagagaagaaagcagctcactccaagtggggaggggcattagcgagcaggaagccaccatcctgtccttggcctgggaagcctgggaaggggtgatagactgtcatggtggaaaagagcaccttcctcgctttccaccagggtgcagggctctgtcctctgaaagtccacagtctgaggtccctgcacctccttcctgttcagggctattttgcccaagagaaaggacacttgggctagtccattaggactcagaagacagtctgacgtctagcactgctctttggtctcccatgttctgccagtgaggctctgcgcgagcattttaatttcttggagtctctattccaaagggcaaatgaagataaccagccctgaagctgctgggaaacacaccagaaaatgacctcacactttattccctgttcgatgaaaaaaaaaaaaatctgagacaatctttgagaacctcttattcaattgttcttaacagtactccataccaaggagagcgtgtgaagaactgtcccactgaccacacgcagtcatatcccatttctcctcacgttggcgcatgccctgcctgcttcttcattaatagcatcatggagataatgaacaaagagaaagagaaataacagtggtttagtgtgttggttggcatttagggaatcctattttttccccagaactcacaagctgaaggaaagaattgaatgctgaatgttgccctctaatctctaactgtggctcccacttcaagaaataaacataatttaaaaaaacactatttttaaagagccctgagagatgatgtaacagtcaagagagcacgcataagacctgagtgaggctcttattatccaccaggccaggagctcacagctgcctgtaactccagatctggggaatcggtcggcctcttctgactcctccaatacccctcacagatgtagatgtggggagtgcatgacgtggtgtgtacatcctgccatgaaggacttgctctgggcccgtaccctcagagaagatggattcttacttctcacctagcaattcatgccatggtttgggaggatgttcagtgtcttctagcccccttacctaggggataagaacatggcttcctgcctttattctgctataagaagtagcaccccagcacccatatgctggggtgcttccctctgcccactctgaatcctgattgcaggttgaggtgtgaattctcctcttgatgctgactggattgaattagcagttcagcttccttagtttccccagcctattgttccgcttatttggaattggcctctatactggatacactcagccttagctttccagtatatgttgctgatctgtaaatatgcagactttgtgggtctttggttttagctatatattttatttttctttttatattttacctatcactctttgaagacgaaggaactcagtaaagtaacattttatagaagaaaatagaaacctgatttccctaagatcttataggtaagaaacacagtgagaccaccagccaggagcacaaagtggatgctccaaccatggcatcagaatgcccttcctttcaaaaagatttgctgtgagctcctctgcaagagcaacagtattcataactactgagctatttctccagcccagacaatggtctcttttttactctggagaatctagaggttcttttatggctgcttaaacagagaatctcatcctgtatgtataaaatgaatcaatagtcaaggtaacagttaatcaggagttgaaaacctgcaacattcacttcttttgtgaatgacattgatttcaagtattggcgacgacaatattcacaaagagcttttaatgctcaagtagcagcaaagatgtaagctatacaccactgtctggggcacaagacacaacgggatttacaacatagatcctactccgagctggccccggaatatctgtttttaaaaatacaatatacgaaatgacaactcaaaggattgggggctgttgtagaaagctatcttccaagctaacagaggatcatcatgactgcgggactactcagtgacgcgggtgctttgggaggggatcactcaaactcttataattgagagacagacagacagacagacagggaaggagggagggagggaggacagctacaggtgtctcctcaggcaccttccacctttggtttgagaagggcgtctcttactgaggcccagatcttctccctgcaggctatagcttgtctgaccagagagccctgggagatactgctgctccctccccagcactgagactgtaggtctatcacacttcttagccttgtgcttgagcttgagctcagaccgtcattctcataaggcaagctctgaaacttccttcacttaatccaagggacgggcaggttctccgcttcagtagcctggcacagtgggtccgggcagtcctgcggtaactaactgtgaagcacagcacaccctcaccttcgtttttttataacctgtaaaacgtcacttagctattacgtggtctgagaaaatgtgggattgtgggcacttctgcagtgatacatattcacatcactaaggacagctgcaggctcttaccgtggtgtccatttccaccaaactgaactcaactgatggagcttcatacatttcagtgttaaataacaaagtaaccttcatggatgcagattagacagcacctcggggtaatgtgcataaaaccaagcacgtatccataggggtaatatcagtatatcagcatgaacagccaaggtggccgtgtgatgactcatcttggctgtcaacttgactgcatctggaaccaactaaaacacaagctgctggcacttctgtgaaggatttttaaaaaaaatatgtatttatttatttatttattttatttaacactgtagttgtactcatggttgtgagccaccatgtggttgctgggatttgaactcaggacttttggaacagcagtcagtgctcttaaccactgagccatctctccagcccctgtgaaggattttcttaatcacattatttgaagcaggaagatgcaccctaaatctgagcagcaccttctggtggcacccagatcaaaggacttggaagaaggaatctgctttctgcctgcttgccctgactctcgctggctgatattccttcactgatattagaaagtgctagcttctttgtgattccaaggtagactaaagaccagaagctcttcaggaattttctaggccttcagtgccagattgagaccatggactgaacaacgtctgattctttacctatccagtgtgagacagccattgttggactatacagaccgtatcatataagtcactcttataaattcttatatatgtatacatatgaaagagagtgtgtgtgtgtttgtatatacttggcccagagagtggcactacttagaggtgtggccttgttgaagtaggtgtgtcactgtgggcatgggcttaagacccttatcctagctgcctggaaagttagtgttccactagcagctttcagataaagatgtagaactctcagctcctcctacaccatgtctacctggatgctgccatgctcctgccttgatgataatggactgaacccctgaacctgtaagccagccccaattaaatgttgtttataagacttgctttggtcatggtgtctgttcacagcagtaaaaccctaagacagaagttggtaccagggactggggtattgcttgatacacctgaccatgcttttgtttagaaaaatgtgggttttgggactttggatttggaaagcagtgggatgctttaaatggggcttaatgggctatcctagtaggaatatggaagactttgttgctgtgagtgatttgaattgtgcagacttggcccaagaggtttcagtgaagaatttcaatatgtggtctagagactgtttttgtgctatttttggtgaagaatgtggctactttttgcccttgtctgaagaatctgcctgaggctaaggggaagagactcagattaattgcattgacaaaggaagtctcagaaacgcgcgccataaactttgttctctggttaagtctcatgaagaacatttcaaacaagcatagtgagctgagaaagggaaaatataaaatatatggttcaagtattaaaggggcaccaggaagggaaaaggagctgaatcctgtgttccaggattaaattgaattaagggagttgtgaccttggggcaagattccacccaactaaatttaggtccaggcatgttagtataaacctttaattccagaaggcaaagacaagcagatctctgagttcaaggccaacctagaacagagcaagttctaggtgaagaaaaacttaaaatatgggcttggtggatcacacctttaattccagtgctcagggcatgcagatctctgagttcaaggtcagtctacagagcaagatccagggcagccaagcttaggcagtgaaggagctggaaaagaggaagctggtgataatgtaatagaacgagggggccatgttccagccccagcaagcagcagaactcagccactttggccatgtggctctggctttagagtcaaaaataaaagggactactgggatattgatgctggttagctggagctaagaaattagtagtgattaagaacagaccagcatcactgaggtgaaatctgggaggtgttttctaagagcacagaggttgtgttccagagatagcgaggttttaccttgtgctggactctacttagtaatgtataagagtcacccaggtggtactggttttgaaggcatgaaggggtcatgaagagcagctgaggctcggcactgtgagaggccatagaaggtcaatggtgaaggtgcagcctcagttgcagctgatggcccaggactgaagggatcatgcaaaggagttgaggcttggcaccatgaagcgaacctatgaaagactattggtgaagcctatttggagtggaacaccccagaattttggagacaccagtaccacaggatgatcactaagaacagcagcagcagtggcgtggatcaacctgagcttagagtgctacagagggcagagctagagaagtgacaccagccctttggaggtcccccggaagatcttgtgtagatcccagacattgaaacaagctgtaacattgaaattgctgtggagaccccaagatgttcaagatgccagagctgtgggatatctggtaaggaatgctgcaaacaagagtggaaccagcccaggagaaagaagtttgttgcagtcaacaaagatgaaaaaggagttggagacctgaaaaccactttgacatcagacatggagatgcagagtttggagtttgcccagctgatttcttgtcttgctttggggattacagttaagtgactggatggatcttagaagagactttgaactttggacttttaacattgttgagactactaaagactatgtggactttggaagttgaactaaatgtacttttttaaattgtgctatggttatatatggcccccatagactcatatgtttgaacaggcgtatgggggccagggagtggaatatgatggtttgtacatgcttggcccaggagtggcactatttagaggtgtggccttgttgaagtaggtgtgtcactgtgggtgtgggcttaagaccctcaccttagctgcctggaagtcagtctcccactagcaatcttcagatgaagatgtagaactctcagctcctcctgcaccatgtctacctggatgctgccatgctcctgccttgatgataatggactgaacccctgaacctataagccagcccctattaaatacttataatacttgccttgatcatggtgtctgttcacagcagtaaaactctaactaagacaaataaatatggatatattcacatatacacaaacatatatatatggttggatcagtttccaatcaattgatttcctctggatacagttttatggcataggagtgtgtgtgtgtgtgtgtgtgtgtgtgtgtgtgtgtgtgtgagtgaaagagagagagagagtgtaccagatttttgtcatttaaaaaatgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcacatttagcatttttaaaggacccagactaatacaagcccccagctgaagggacaccagtctgaataggcaagatcaagacaaagacaatcacaggccagctagtttccagcaactgtgaagtgtcatattgtagaacatgtccttgcttcaaaaatgctaaatgtgacattttttaaatgacaaaaatctggtacacacacacacacacacacacacgtactcccatgccataaaactgtatccagaggaaatcaattctcacaaaaggtcttgtgattgctgttcccatcctgacaacctcgagcacactccacagtgggatcaccatcagtcggctacttgacctagttttacaaactgtgctgtccccaaagcacacttctcataaatggaaataattaaagtgtgggcactctgaaggactccccttccactcagcacacaccctgctggggagttgagagcatcctggacctatgtcctttccatccgggtgaggtagcgcaggctcattgtctcagactgactgctggaagtgtctgcactgccagggtgaagccaatacctgaactctcttagaaacaagaacagggcaatcattggactctaggtgggtcctgtgggatccacacttcaggtcagcataatatctcctgactctgagcactggaaacacgcaccaatgatccaggcttcttaactctagtcaaagctgaggatgcaggctcgtctgcagagtgcttgcctaacgtgcagaaagcccaggttcaactttatagaaaccagtgatggtgttgcacatctacaatcccagccctctggaggtagaggccagaggacaaacgttcaaggccatcctgaaatagactcccagaccttagcatatctgttgccattggagataccattctgaccttagaacccagcacctgccaaaatggaaaccaagacctaatccatcaaagacctggtggatagctacaagttccaggaaaacatctgaatgtgttaaccttgcctattggtttctgtagctttgcttcttgctaacagaagtacgccaaacagaatgtggttttgttcataaaagacaaagaccatgaggcttggaactgcatgatttgggcaagttctccaagcagttgctggccagcaatacacactttctatttgactcttttttttttttttaacttgtcttacacgtagagtttgaggctagcctgggcttcataagatcctgtttatgaacaaaccaaccctttttttttttaatttaatttttctaaagtgccaccagctgggatcgagtgttcaagcacattagcctacaaaggacacttcacactcaaagttcaagtctagcctacaacacggtctcaggtagtgcccaagaagaaagggaggtttccaccatggggaaggtggaagggtatcaattccccaaagctgaaatttacctctatgtatagtatgttgggctcctcccaacaaagagcacactgggagtcacggggccatagaagtcaggccaacctataggggagagctgtggtagggcactcatgggctccgaagcctctagggtcgtgaatgcaggcctgcatgtgaccaccattttctcccactcacagtgtggtgaggttctttctaaagcctggaagcaaatatccaggaaaaagaaatataaccacatctctgaagacagcagcaccttctctcgggttctttaggcccatctgtgtcctctccaggcaggcagactgcctgtaaatcccagggctccggctgcaaactcacccactcctggctctggatcttctcggctgtagcacagcacatgcgtctccttttgcagcctggcacctcttgcagtaacaagacttgccaagaagtagctacactccctttcctgctctttacatccataatttcgcttttaaaatagcatcttattatacaaagggtcgtccaccaggagcctgaaatagccagagagtttagagctctggtggacttgaggggtttgtgtagtatccctctcttcctttacctcaactgagtcagaatggaggatgagtggctgggagccttgggatgcctttctaaactgagagcttccagagagcctctgtgctcttctccctcccctgagcattgttttgacatcagactttgttcagagaacggcagtacttggggaaatggcagaaaatgcaaattctcccatcctcagggcttctgatccactaaagatttatcctgtcaatgaaagacacagctctcacctcgtgagtggccatggcttgggaacagggactcaggtttccctaactctctcaacatgagactctgggaacattcagatttcttggggtccattatttcaaaacttaagagctaaagggagagggccaggtctccctttagagcactgtcattcctgccagcccaggtccccctttagagcactgtcattcctgccagcccaggtctcccttagagcactgtcattcctgccagcccaggtcccccattagagcactgtcattcctgccagcccaggtccccctttagagaactgtcgttcctgccagcccaggtctccctttagagcactgtcattcctgccagcccaggtctcccttagagcactgttattcctgccagcccaggtccccctttagagcattgtcattcctgccagcccaggtccccctttagagcactgtcattcctgccagcccaggtctcccttagagcactgtcattcctgccagcccaggtctcccttagagcactgtcattcttgccagcccaggtccccctttagagcactgtcattcctgccagcccaggtctcccttagagcactgtcattcctgccagcccaggtccccctttagagcactctcattcctgccagcccaggtctcccttagagcactctcattcatgccagcccaggtctcccttagagcactgtcattcctgccaacccaggtctcccttagagcactgtcattcctgccagcaggcagcgaagctctccaggattctgacctcctccccagccgcaggtctgcttgacaaactgaggcagggagcaagactataaaaccaagattcttaatcaaaggctgagcttctccttttcctcttgactccaagctccaaactcttacataagcaacacaatctggcagacaaaaatcattctctcactcctctgcaagtccttggcagccctgggtacggcagccggaagttcctcacccatctggaggcaggatgctgccagccatccccacagaagctaataaactccttccttcacccatgtcccaggtgaaagtattgcctagatgcacatgtgcatacacacacacacatgcacacgcgcgcacgcatgtacacacacacatacacatacatgcacacccactcgagcacacacatgtacaggtatgcacactcatgtgcacattcttctccagctgctggtttgtaacagtgtgtggctctgtgcagtttcaggcctgccctaaagagcagcctaggttgttaagtttaggactaatcaggccatgcctacccgtcctaacctttctttcgggcactgtaaaataattgcaaatccctcacctgtgagcctcaaacaccctaacatgtccccccagttccaggagaataataggcccttatttaattttcacttccctaaggaagtttccaggctggggctgcctgtctggacacctgagtgctgccgacagagggtgtcttagtccctgtcctacagcggtgaagagacaccacaaccaaggcagctcttataaacaagagcactttactgtgtattgctcacagtttcagaggtttagtccattgtcatcgtggtaaggagcttagcagtacacaggtgggtgctgtggagcagtaactgagttataatccaatccattggtgaaggaagacactgagcctaccatgggcttctgaaacttcaatggccactcacagtgacacactttctccaacaagatcacactttctaatccttgtgatcctttcaaatggtcccacttcctatactgacgaagatttcaaatgtatcagcctgtggggaccattcttattcaaaccaccacacatgggtccatctcttccttacttccctacgtgtcagccaagtatttgttcccagaatggagatgccacctatgaatcagagatagccaggctgaagcagagctaggctggaacctctcccttcctctgagacctgtaatcagggagccctactctgcactggggcctgaccactcctcagagcccctaccctcctgctgtctgcctttggctttaataactcttctactaaatgtcttattcagaaggacacaagccttacaatggataactgttccagtttcattctgatgctgtgaatatatatatatatatatatatatatatatatatatatatatatatacaccctaatgataagcagcttagggagaaaggggttcatttggcatgcaatttcaggttatggtccattgtggaggggtgtggaggggaagttaagacaggagcctgaagcaggtcacagctcatccacagtcaagagcagagatcaatgcacacaccattgcttgcttgtgctcggctcaatttcttcatgcttacgctgttcaggatccctgcctagggaatggtgccacccatggtggacttggtcttcccatatccgttaataaccaatacaatccccacagacatagcaacagatcaacctgctctagataattcttcagttgaggttcccttcccaagtgacttgaggttgtaccaagctgatggctaaaaattaacccacagaatggttaagtcactgttcctgtattgggacacattggtgaaacacttctataaaagaaataggtactgaggacattctatacattaagctctatgggaaattaaggtaaaatctgataaacactgcatttacagtgtcagacaggtgaggctgttctgccccgggtccctcggatgcccctcaggccttagaatggcaatgtctgtgcctcctcccagttgtttgcagccatgggtctctcagaagagctggatcatggacaccctagagactgctctcagagggggcaggctggagacggtgagcaccccggctctctctttccctcaaggacataaaaaacatttacatattgccctccctgtaccccagcacaacaagcctcacatatgtgtccccttctcactgtccgttacacctgtgggtgtaaaaacactgttctgtcctcaaggacgtgagagagggtttattctggagcaaagatgaatgaccacgggctctgggctctgtctagggaatgcagattcagatcacagtgtcagaacaaacactgcagacgcattgctggggaacactgggtaggcagttcagagccaagcaggaaagtttctgcatagacctcggctgctacctgatggcatccttagcctttggttggtggaagctagaagtcggtcaggttaatataccccaagtatttttacctgtcagtcacaaggacgttaggtcagatgcagggaggtgggtgagatgaatggggctctgaatgatggcctagcggaaacataacagggctctggaccttcaacaccatcactgtcatcctgctcaggtgtcagcttggcagaaggtttggtggaaggtaccgctctcccaggaacttgttcacaggctctccctggggttttccccattcactgttgcttgaatgaactggattctcttcaaagtctccccagattgaggattcctcgcttccacaccagcttcatagaaatcccagggaaacaacaaaataagcttctgtggttgtttttgtttgagacgggtttcatgtaacccaggctgtgtaacccaggctggccacacacatgtacatgatcacacacatacgcacacacacacacatgctatataggcaaggaagaccttgaacccccgatcttcccagcttcgtctctctaatgctggggctgtgggtgtccatcagcacacctggcatgaacaactgaaagcttttctaacttgtgggagttttctatgtcaagcactgaccatgttttaaaattccctccccactgacatactgagtgggggggggcactttggaagtgaccaggtcacggatgcttctgctaatgtgaaatgtgccctttaagaatagactatcttgggccagagagatggcgcagctgtcaagagcactcgttgcttttgcagaggacctgtgtttgattcctacatggcggttcacaaccatctgtaacttcggttccagaccatatgacatcctgtcctgacctccttaaccaccaggcactcacgtggcacacagatatacctgcaggcaaaatactcataatcataaaacaaaaataaaaaaataaaaaaacagacttccatctgtcacctgaggtacagcaggtagctgtagccaagcagagtcctcagcaggtcccacacctttctggtcctggaattcccagcctctggcttccaggaactcactttctgctcagccactggtctatggtgttttgtcctaccagcttagatgactaacagacaaggctgccttcctggctgtcaaggaggtggctgtgagctactttccaaggggaatgtcagagtccagaagcaaagaactcactattcacgtgtactggttctttctcccttgcctcttccctcagtcctgatttctgggatccatccaaggtcatcttccacccctagatgtctgtcctgggttctgcctctgtaggaacccaaatggaggcaaagtatcctaagggagctctcacaggaaaagtgggcactgcccccaaagccaggactgctcccttctttcttgttaagttatttcagataaagtctgatgaccatgcccaaggatcttataaaagtgtcagggtatagtcaggccagtattctgaatcgtggccagaggaactctgccttgcagcaaaggtggcagagcatacctttccacactaggcattttttcctgaggtgaacagaaaccctgataaggactgagtacacgaaagtgtaactgtctctgagtcctactaggttgtgtgagcctgtgctcacatccaccactatgacactggacaactctctgggcccaggtttatgtcaatgtggctgcagatgtagggcaggataaaacaagagcctgctgggcagggtcatgcagccactccgagtcacccacactctcaataaacccaataacttcagttatttcgccaagctggactggacagaggctctctttggtcggtctgcaccccctttactgggatgaatatgagactcaccagacaggggtttggatgtgaatgaccgaggtaaacctgaacctgtagtcagcagccaaatctcccccagaggtcagcacatcccctgttaagtgcctcgaagctgcagttgggttgtgactaagctccatcagaaaggacggaccagttgctcagtcagcacaggtgtgtgctgaccaaaggtcgccacccttgttataatatcttttacatcacagtttagacctctatcccttgtggggttttctagagaatcatgagaagaaatatcctacaaaacagatcttgtttacatgatttgggagaggaagggggtcattttcctccatacacatgacacttacatcataaatattcattaaaaaacacctttgaaagccatggtggaaaagtcactgtgctctgcctactcgtggcacagcccagcccagtaaacatttccctcagcttcactaagtacgcttcttacactgtctctttttccaactggatcccttccctctagaagataggagttggaagacccacgtaataaacagacacttgatttgtgtcccaggaaaccacacaacaaattaccatttctaaaatacgttcaggagaaaagaagccctcaagacctaaaacagacgcctttcctaaaaccgatgccttcgtggatacagcagacagcttcttgacctccactaaacaggatgagcggtggcaagcagaaatccaatgaataataaatcaggttcctgcttgtccctcgatgcctcactaagcttcctacactgtgaaatggactcgtgttgttgagaggcctctcatgactggaaaagccctttgttgaaaatgtcatgaatttcccagcactgaatgagatatcatttaacattataattttttaaagatccatttgtcttattttatgtatgcaggtgttctgcatggatggatggatggatgggtgggtggatggaaggatggatggatggatgggtggatggatggatggatggatgggtgggtggcacatgcatgcagtgcctgaggaatcagaagagggtgtcagatcccctgcaactggggtaacagttggctgtgagccaccatgtggattctgggaagcaaacctggctcctctgtaagagcaacaagcactcttaactgctcagccatctctccagtttgtccttcaacattttaatcaccaaaaccagaattaaattcctagattcccaaatcggccaacagtcagatttctcctcgtcacttcttccttttttaattagatattttctttattttcacttcaaatattatcccctttcctagtttcccccctgaaaatcccctatctcctcccccctccccctgctccccagcccacctacccactcccattcccagtcctggccttcccctatactggagcatagaaccttcacaggaccaagggcctctcctcccatcgatgaccgaataggccatcctcagctacatatgcagctagagccacaagttccaccatgtgttttctttgattggtggttttggttccaaggagctctgggggtactggttagttcatattgttgttcctcctatggggcttcagaccccttcaactccttgggtactttctctagctccttcattggggaccttgtgctcctggcaaatacagaagtgaatgctcacaatcgtccattggacggacagagcacaaggttactttttcctttcaaatataacatccggacaagatgttggcagtcctccattggggacagaaagggtccaccaaggaggagttctgggctcttaatgtggagtctggggcaggggtggggcgcagtggagactacatagtggacactacctttttcatcctgttccagagagcaactcgagggaggacttattttggtttgtggttaaagggagacaggccatcatggtggggaagactaacagcagggagctttgtgacagcagcagaatagggctggacctcctacctgctcacacctccgtgggacaggaagccaagacagcaagtgaggcctggcaatcaacctccaggcctgctccaatgagccactttctcccataaggctgtagcttctaagggttccacaacctctcagaacaaccccactagctgggaccaagtgttctaacatgagagagaatctcacattcaaacaaatctgctaccatctctcagcagatgaaacacaatacaacccacaaccaagacacaaccccttctttatgtgggcagataagctatcccccttcccttctaagcatacagggagctcacacaccaaagatccccgagaagggtcctccagcaggccttcctatgcattcaagcaaacttgccacccagccatctccaacctctgcccacttccgtgtacccatccaggaccatcacacatgcaacttatcttccctgcttgctgtctttctcttccacatctctcctgctacagtggatgtgtatattgttcaccgtgaccctctacggcacctgcagacagctggtgttctcagcatggttatactgagatgagactgatggggcgagaggtgatctcagggaacagaagtgaggaagtgaggaccaggagaagctagttagagatgctatcagggttactgctggagcaaaggacctttgagaagtttccagaagtttctagaatgttccattgcagaactgggtttttccatccttactggcttaggttaggcctctggggagcaacatcgtcatttataggaataaagcttgaaggaagagaagtggagttccagtgtatatgtgtgtgtgtgtgtgtgtgtgtgtgtgtgtgtgtgtgtgtaaaattagagtggggagaggaacatagagcaaggacattgtctcaaaaatcaactgcttcaggggcattaaactaatacaagagaaacgcaagccccagcctctcttgctgctaaaggtcatactcagaattactgttcctgccgggcatggtggtgcacgcctttaatcccagcactcgggaggcagaggcaggtagatttctgagttcgaggccagcctggtctacaaagtgagttccaggacagccagggctatacagagaaaacctgtctcgaaaaacaaaacaaaacaaaacaaaacaaaacaaaattactgttcttgcctcagctgcttggaggaacctcactgaatcactatggccatagaatccaggctccacctattttccatcagtcctacccagaattcccagggaagatgaagaaaccatggctacaatattttattaaataaagagtccattgtgccaccttcttccagcttctaactgtccttgacacctttggcttctcctggtggcttttctactctggcctttgaccctgcctgtggtcaggtgctggacccttctcccgttccttcctctggctgtcacacctgggaatctgggcagagatgccttttctcttccctcgcttcccaccttttgagtctcttcctacagcttccaggtgggaagccaccatctacagaaagagtcttgggttgtacctccagtgtccctctgtccaggacttgagatcacaaccttctctagctgtccacccataacctggattcatcccccacgccccgccagacacacaccagttggtgtgttttcttctgaactccccctgtggccaatacactcccacccacccttgtctctcagcccagaacttcacagtcagccccagacacggagtacctttgggtttcttgaagagcaagatgctgcttccatgcctggaagtttctgcttatgtctactgttctagctattcggatgttctattgtaggcacttaggactcgtatagtgtctgctacatttcatgatagtgaataaatttcacaaaagcatattaggcttcctataaatctgtcctcattcctcaatggccctcccatctcctgtcttcctaacctgtcctggtctcccgtgtctgccctagggacacctccatgtcaccaccagactcagtggaaatccttactccgcccttggctattagtgcagatctgaactcagttccttgtacttgcaggcaagcacttcactggctgagccgcctcccagttccagctccaggcccccggctcccaaaggtgtttatttgtgtgggtatttattttgtccagtcttggtcacaccgcatcctcaggggctgggcacagttcataaatcttgaggcaagcgatggagggaaggaggcagctaagcgtccatatctcagccaccgacccagggaagtcagcgcctgtggattctgaccatatcgaacagccttgtgcccagctgctttatccacaattccggacatgctcgatctgtcacagatacattcccacaacctgagctgtcttgtgcgggaaatcaccccacagcatttaatctgttgctgtttaaaacatgttgcctctaggttgcagacaccgctagagccacaaccatgaacctaaactcttggcatcacttgctgtttctcatagtccccctcagccggaagtccccaaactgtgtgccttttctatttagaaagagtttctaaccctttctccattcaccctagcttgacagggttgagggcatggttgccctggctggtggtgaccccaagttacaagctagcagcaaggaggttgctgtggggcttcctcagtatgtgttctgtggaatggggttagagggattcagcaaattctagcaccctgggcatagataatcactttgttatgtgagaactgggggttgcaggattgtgcgcactacagcagagagagccccctctctccttcttgcttggtaagagtctttttctcagccaagatcctcatcacccagcgaaatcccataactttagagggactagactggaaagggtgatctgagctcttgggaaggtgcgagcccagcccgcatggctcagccagccagagcttgggagtgcctgagacactctctggcgccacttcacgaccaaaagcatcagtagatgataggcccctgggaagtcgtcgtggaaagaaattacaaatctttttcccagaggcttttcgcagaaaggcaggagctgcacccgatcttacaattgtgtaagaatagaatccaggatgccaactgcaattgagttctgaaaaattgggagcccgatttccctctcttacttgtgagagcccactcaggtctgaggtggtcccagagaacacaccaggattacatctgctgacacccagcctgtgagggtcccccagtttccttgaaggatttgatccccaaagctcactgaacttggtcagcttctccattgcagataaactcctgtttttcaccgagagtggaggtggcaccctccctgaggtggactctgcacaggcgccgaacaggtgggaaggaagctctttagataaagagtaagacccatgcaaagtgcccccctgggaggggctatcctcattcactgggacgcttcccttctctccggagggccacatcaatcggtggtccctccagtggctgcctctgagcacgtgtcctgctggactgcgtcagcactgggtaaacagatgactggctgcgaaccgggaggagctatttaagagcagtcaccctcccgcctgccctcaacttagctggactgcagccttctccgctggaactcgccaagccagctgatttccccatccaaaggtaagtagctggctgatccaacaatcttgagtgtgaagaatattggcctgcagctctcgaggatatcaagatctggcctcggcggccaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 2 - DNA, 17797 bp, Mus musculus
ggcctaactggccggtacctgagctcgtgccaccgtgccaccagctgggatcgagtgttcaagcacattagcctacaaaggacacttcacactcaaagttcaagtctagcctacaacacggtctcaggtagtgcccaagaagaaagggaggtttccaccatggggaaggtggaagggtatcaattccccaaagctgaaatttacctctatgtatagtatgttgggctcctcccaacaaagagcacactgggagtcacggggccatagaagtcaggccaacctataggggagagctgtggtagggcactcatgggctccgaagcctctagggtcgtgaatgcaggcctgcatgtgaccaccattttctcccactcacagtgtggtgaggttctttctaaagcctggaagcaaatatccaggaaaaagaaatataaccacatctctgaagacagcagcaccttctctcgggttctttaggcccatctgtgtcctctccaggcaggcagactgcctgtaaatcccagggctccggctgcaaactcacccactcctggctctggatcttctcggctgtagcacagcacatgcgtctccttttgcagcctggcacctcttgcagtaacaagacttgccaagaagtagctacactccctttcctgctctttacatccataatttcgcttttaaaatagcatcttattatacaaagggtcgtccaccaggagcctgaaatagccagagagtttagagctctggtggacttgaggggtttgtgtagtatccctctcttcctttacctcaactgagtcagaatggaggatgagtggctgggagccttgggatgcctttctaaactgagagcttccagagagcctctgtgctcttctccctcccctgagcattgttttgacatcagactttgttcagagaacggcagtacttggggaaatggcagaaaatgcaaattctcccatcctcagggcttctgatccactaaagatttatcctgtcaatgaaagacacagctctcacctcgtgagtggccatggcttgggaacagggactcaggtttccctaactctctcaacatgagactctgggaacattcagatttcttggggtccattatttcaaaacttaagagctaaagggagagggccaggtctccctttagagcactgtcattcctgccagcccaggtccccctttagagcactgtcattcctgccagcccaggtctcccttagagcactgtcattcctgccagcccaggtcccccattagagcactgtcattcctgccagcccaggtccccctttagagaactgtcgttcctgccagcccaggtctccctttagagcactgtcattcctgccagcccaggtctcccttagagcactgttattcctgccagcccaggtccccctttagagcattgtcattcctgccagcccaggtccccctttagagcactgtcattcctgccagcccaggtctcccttagagcactgtcattcctgccagcccaggtctcccttagagcactgtcattcttgccagcccaggtccccctttagagcactgtcattcctgccagcccaggtctcccttagagcactgtcattcctgccagcccaggtccccctttagagcactctcattcctgccagcccaggtctcccttagagcactctcattcatgccagcccaggtctcccttagagcactgtcattcctgccaacccaggtctcccttagagcactgtcattcctgccagcaggcagcgaagctctccaggattctgacctcctccccagccgcaggtctgcttgacaaactgaggcagggagcaagactataaaaccaagattcttaatcaaaggctgagcttctccttttcctcttgactccaagctccaaactcttacataagcaacacaatctggcagacaaaaatcattctctcactcctctgcaagtccttggcagccctgggtacggcagccggaagttcctcacccatctggaggcaggatgctgccagccatccccacagaagctaataaactccttccttcacccatgtcccaggtgaaagtattgcctagatgcacatgtgcatacacacacacacatgcacacgcgcgcacgcatgtacacacacacatacacatacatgcacacccactcgagcacacacatgtacaggtatgcacactcatgtgcacattcttctccagctgctggtttgtaacagtgtgtggctctgtgcagtttcaggcctgccctaaagagcagcctaggttgttaagtttaggactaatcaggccatgcctacccgtcctaacctttctttcgggcactgtaaaataattgcaaatccctcacctgtgagcctcaaacaccctaacatgtccccccagttccaggagaataataggcccttatttaattttcacttccctaaggaagtttccaggctggggctgcctgtctggacacctgagtgctgccgacagagggtgtcttagtccctgtcctacagcggtgaagagacaccacaaccaaggcagctcttataaacaagagcactttactgtgtattgctcacagtttcagaggtttagtccattgtcatcgtggtaaggagcttagcagtacacaggtgggtgctgtggagcagtaactgagttataatccaatccattggtgaaggaagacactgagcctaccatgggcttctgaaacttcaatggccactcacagtgacacactttctccaacaagatcacactttctaatccttgtgatcctttcaaatggtcccacttcctatactgacgaagatttcaaatgtatcagcctgtggggaccattcttattcaaaccaccacacatgggtccatctcttccttacttccctacgtgtcagccaagtatttgttcccagaatggagatgccacctatgaatcagagatagccaggctgaagcagagctaggctggaacctctcccttcctctgagacctgtaatcagggagccctactctgcactggggcctgaccactcctcagagcccctaccctcctgctgtctgcctttggctttaataactcttctactaaatgtcttattcagaaggacacaagccttacaatggataactgttccagtttcattctgatgctgtgaatatatatatatatatatatatatatatatatatatatatatatatacaccctaatgataagcagcttagggagaaaggggttcatttggcatgcaatttcaggttatggtccattgtggaggggtgtggaggggaagttaagacaggagcctgaagcaggtcacagctcatccacagtcaagagcagagatcaatgcacacaccattgcttgcttgtgctcggctcaatttcttcatgcttacgctgttcaggatccctgcctagggaatggtgccacccatggtggacttggtcttcccatatccgttaataaccaatacaatccccacagacatagcaacagatcaacctgctctagataattcttcagttgaggttcccttcccaagtgacttgaggttgtaccaagctgatggctaaaaattaacccacagaatggttaagtcactgttcctgtattgggacacattggtgaaacacttctataaaagaaataggtactgaggacattctatacattaagctctatgggaaattaaggtaaaatctgataaacactgcatttacagtgtcagacaggtgaggctgttctgccccgggtccctcggatgcccctcaggccttagaatggcaatgtctgtgcctcctcccagttgtttgcagccatgggtctctcagaagagctggatcatggacaccctagagactgctctcagagggggcaggctggagacggtgagcaccccggctctctctttccctcaaggacataaaaaacatttacatattgccctccctgtaccccagcacaacaagcctcacatatgtgtccccttctcactgtccgttacacctgtgggtgtaaaaacactgttctgtcctcaaggacgtgagagagggtttattctggagcaaagatgaatgaccacgggctctgggctctgtctagggaatgcagattcagatcacagtgtcagaacaaacactgcagacgcattgctggggaacactgggtaggcagttcagagccaagcaggaaagtttctgcatagacctcggctgctacctgatggcatccttagcctttggttggtggaagctagaagtcggtcaggttaatataccccaagtatttttacctgtcagtcacaaggacgttaggtcagatgcagggaggtgggtgagatgaatggggctctgaatgatggcctagcggaaacataacagggctctggaccttcaacaccatcactgtcatcctgctcaggtgtcagcttggcagaaggtttggtggaaggtaccgctctcccaggaacttgttcacaggctctccctggggttttccccattcactgttgcttgaatgaactggattctcttcaaagtctccccagattgaggattcctcgcttccacaccagcttcatagaaatcccagggaaacaacaaaataagcttctgtggttgtttttgtttgagacgggtttcatgtaacccaggctgtgtaacccaggctggccacacacatgtacatgatcacacacatacgcacacacacacacatgctatataggcaaggaagaccttgaacccccgatcttcccagcttcgtctctctaatgctggggctgtgggtgtccatcagcacacctggcatgaacaactgaaagcttttctaacttgtgggagttttctatgtcaagcactgaccatgttttaaaattccctccccactgacatactgagtgggggggggcactttggaagtgaccaggtcacggatgcttctgctaatgtgaaatgtgccctttaagaatagactatcttgggccagagagatggcgcagctgtcaagagcactcgttgcttttgcagaggacctgtgtttgattcctacatggcggttcacaaccatctgtaacttcggttccagaccatatgacatcctgtcctgacctccttaaccaccaggcactcacgtggcacacagatatacctgcaggcaaaatactcataatcataaaacaaaaataaaaaaataaaaaaacagacttccatctgtcacctgaggtacagcaggtagctgtagccaagcagagtcctcagcaggtcccacacctttctggtcctggaattcccagcctctggcttccaggaactcactttctgctcagccactggtctatggtgttttgtcctaccagcttagatgactaacagacaaggctgccttcctggctgtcaaggaggtggctgtgagctactttccaaggggaatgtcagagtccagaagcaaagaactcactattcacgtgtactggttctttctcccttgcctcttccctcagtcctgatttctgggatccatccaaggtcatcttccacccctagatgtctgtcctgggttctgcctctgtaggaacccaaatggaggcaaagtatcctaagggagctctcacaggaaaagtgggcactgcccccaaagccaggactgctcccttctttcttgttaagttatttcagataaagtctgatgaccatgcccaaggatcttataaaagtgtcagggtatagtcaggccagtattctgaatcgtggccagaggaactctgccttgcagcaaaggtggcagagcatacctttccacactaggcattttttcctgaggtgaacagaaaccctgataaggactgagtacacgaaagtgtaactgtctctgagtcctactaggttgtgtgagcctgtgctcacatccaccactatgacactggacaactctctgggcccaggtttatgtcaatgtggctgcagatgtagggcaggataaaacaagagcctgctgggcagggtcatgcagccactccgagtcacccacactctcaataaacccaataacttcagttatttcgccaagctggactggacagaggctctctttggtcggtctgcaccccctttactgggatgaatatgagactcaccagacaggggtttggatgtgaatgaccgaggtaaacctgaacctgtagtcagcagccaaatctcccccagaggtcagcacatcccctgttaagtgcctcgaagctgcagttgggttgtgactaagctccatcagaaaggacggaccagttgctcagtcagcacaggtgtgtgctgaccaaaggtcgccacccttgttataatatcttttacatcacagtttagacctctatcccttgtggggttttctagagaatcatgagaagaaatatcctacaaaacagatcttgtttacatgatttgggagaggaagggggtcattttcctccatacacatgacacttacatcataaatattcattaaaaaacacctttgaaagccatggtggaaaagtcactgtgctctgcctactcgtggcacagcccagcccagtaaacatttccctcagcttcactaagtacgcttcttacactgtctctttttccaactggatcccttccctctagaagataggagttggaagacccacgtaataaacagacacttgatttgtgtcccaggaaaccacacaacaaattaccatttctaaaatacgttcaggagaaaagaagccctcaagacctaaaacagacgcctttcctaaaaccgatgccttcgtggatacagcagacagcttcttgacctccactaaacaggatgagcggtggcaagcagaaatccaatgaataataaatcaggttcctgcttgtccctcgatgcctcactaagcttcctacactgtgaaatggactcgtgttgttgagaggcctctcatgactggaaaagccctttgttgaaaatgtcatgaatttcccagcactgaatgagatatcatttaacattataattttttaaagatccatttgtcttattttatgtatgcaggtgttctgcatggatggatggatggatgggtgggtggatggaaggatggatggatggatgggtggatggatggatggatggatgggtgggtggcacatgcatgcagtgcctgaggaatcagaagagggtgtcagatcccctgcaactggggtaacagttggctgtgagccaccatgtggattctgggaagcaaacctggctcctctgtaagagcaacaagcactcttaactgctcagccatctctccagtttgtccttcaacattttaatcaccaaaaccagaattaaattcctagattcccaaatcggccaacagtcagatttctcctcgtcacttcttccttttttaattagatattttctttattttcacttcaaatattatcccctttcctagtttcccccctgaaaatcccctatctcctcccccctccccctgctccccagcccacctacccactcccattcccagtcctggccttcccctatactggagcatagaaccttcacaggaccaagggcctctcctcccatcgatgaccgaataggccatcctcagctacatatgcagctagagccacaagttccaccatgtgttttctttgattggtggttttggttccaaggagctctgggggtactggttagttcatattgttgttcctcctatggggcttcagaccccttcaactccttgggtactttctctagctccttcattggggaccttgtgctcctggcaaatacagaagtgaatgctcacaatcgtccattggacggacagagcacaaggttactttttcctttcaaatataacatccggacaagatgttggcagtcctccattggggacagaaagggtccaccaaggaggagttctgggctcttaatgtggagtctggggcaggggtggggcgcagtggagactacatagtggacactacctttttcatcctgttccagagagcaactcgagggaggacttattttggtttgtggttaaagggagacaggccatcatggtggggaagactaacagcagggagctttgtgacagcagcagaatagggctggacctcctacctgctcacacctccgtgggacaggaagccaagacagcaagtgaggcctggcaatcaacctccaggcctgctccaatgagccactttctcccataaggctgtagcttctaagggttccacaacctctcagaacaaccccactagctgggaccaagtgttctaacatgagagagaatctcacattcaaacaaatctgctaccatctctcagcagatgaaacacaatacaacccacaaccaagacacaaccccttctttatgtgggcagataagctatcccccttcccttctaagcatacagggagctcacacaccaaagatccccgagaagggtcctccagcaggccttcctatgcattcaagcaaacttgccacccagccatctccaacctctgcccacttccgtgtacccatccaggaccatcacacatgcaacttatcttccctgcttgctgtctttctcttccacatctctcctgctacagtggatgtgtatattgttcaccgtgaccctctacggcacctgcagacagctggtgttctcagcatggttatactgagatgagactgatggggcgagaggtgatctcagggaacagaagtgaggaagtgaggaccaggagaagctagttagagatgctatcagggttactgctggagcaaaggacctttgagaagtttccagaagtttctagaatgttccattgcagaactgggtttttccatccttactggcttaggttaggcctctggggagcaacatcgtcatttataggaataaagcttgaaggaagagaagtggagttccagtgtatatgtgtgtgtgtgtgtgtgtgtgtgtgtgtgtgtgtgtgtgtaaaattagagtggggagaggaacatagagcaaggacattgtctcaaaaatcaactgcttcaggggcattaaactaatacaagagaaacgcaagccccagcctctcttgctgctaaaggtcatactcagaattactgttcctgccgggcatggtggtgcacgcctttaatcccagcactcgggaggcagaggcaggtagatttctgagttcgaggccagcctggtctacaaagtgagttccaggacagccagggctatacagagaaaacctgtctcgaaaaacaaaacaaaacaaaacaaaacaaaacaaaattactgttcttgcctcagctgcttggaggaacctcactgaatcactatggccatagaatccaggctccacctattttccatcagtcctacccagaattcccagggaagatgaagaaaccatggctacaatattttattaaataaagagtccattgtgccaccttcttccagcttctaactgtccttgacacctttggcttctcctggtggcttttctactctggcctttgaccctgcctgtggtcaggtgctggacccttctcccgttccttcctctggctgtcacacctgggaatctgggcagagatgccttttctcttccctcgcttcccaccttttgagtctcttcctacagcttccaggtgggaagccaccatctacagaaagagtcttgggttgtacctccagtgtccctctgtccaggacttgagatcacaaccttctctagctgtccacccataacctggattcatcccccacgccccgccagacacacaccagttggtgtgttttcttctgaactccccctgtggccaatacactcccacccacccttgtctctcagcccagaacttcacagtcagccccagacacggagtacctttgggtttcttgaagagcaagatgctgcttccatgcctggaagtttctgcttatgtctactgttctagctattcggatgttctattgtaggcacttaggactcgtatagtgtctgctacatttcatgatagtgaataaatttcacaaaagcatattaggcttcctataaatctgtcctcattcctcaatggccctcccatctcctgtcttcctaacctgtcctggtctcccgtgtctgccctagggacacctccatgtcaccaccagactcagtggaaatccttactccgcccttggctattagtgcagatctgaactcagttccttgtacttgcaggcaagcacttcactggctgagccgcctcccagttccagctccaggcccccggctcccaaaggtgtttatttgtgtgggtatttattttgtccagtcttggtcacaccgcatcctcaggggctgggcacagttcataaatcttgaggcaagcgatggagggaaggaggcagctaagcgtccatatctcagccaccgacccagggaagtcagcgcctgtggattctgaccatatcgaacagccttgtgcccagctgctttatccacaattccggacatgctcgatctgtcacagatacattcccacaacctgagctgtcttgtgcgggaaatcaccccacagcatttaatctgttgctgtttaaaacatgttgcctctaggttgcagacaccgctagagccacaaccatgaacctaaactcttggcatcacttgctgtttctcatagtccccctcagccggaagtccccaaactgtgtgccttttctatttagaaagagtttctaaccctttctccattcaccctagcttgacagggttgagggcatggttgccctggctggtggtgaccccaagttacaagctagcagcaaggaggttgctgtggggcttcctcagtatgtgttctgtggaatggggttagagggattcagcaaattctagcaccctgggcatagataatcactttgttatgtgagaactgggggttgcaggattgtgcgcactacagcagagagagccccctctctccttcttgcttggtaagagtctttttctcagccaagatcctcatcacccagcgaaatcccataactttagagggactagactggaaagggtgatctgagctcttgggaaggtgcgagcccagcccgcatggctcagccagccagagcttgggagtgcctgagacactctctggcgccacttcacgaccaaaagcatcagtagatgataggcccctgggaagtcgtcgtggaaagaaattacaaatctttttcccagaggcttttcgcagaaaggcaggagctgcacccgatcttacaattgtgtaagaatagaatccaggatgccaactgcaattgagttctgaaaaattgggagcccgatttccctctcttacttgtgagagcccactcaggtctgaggtggtcccagagaacacaccaggattacatctgctgacacccagcctgtgagggtcccccagtttccttgaaggatttgatccccaaagctcactgaacttggtcagcttctccattgcagataaactcctgtttttcaccgagagtggaggtggcaccctccctgaggtggactctgcacaggcgccgaacaggtgggaaggaagctctttagataaagagtaagacccatgcaaagtgcccccctgggaggggctatcctcattcactgggacgcttcccttctctccggagggccacatcaatcggtggtccctccagtggctgcctctgagcacgtgtcctgctggactgcgtcagcactgggtaaacagatgactggctgcgaaccgggaggagctatttaagagcagtcaccctcccgcctgccctcaacttagctggactgcagccttctccgctggaactcgccaagccagctgatttccccatccaaaggtaagtagctggctgatccaacaatcttgagtgtgaagaatattggcctgcagctctcgaggatatcaagatctggcctcggcggccaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct